(19) United States
(12) Patent Application Publication
(10) Pub. No.: US 2010/0113892 A1
(43) Pub. Date: May 6, 2010

(54) METHOD FOR DETERMINING PERSONALIZED NUTRITION AND DIET USING NUTRIGENOMICS AND PHYSIOLOGICAL DATA

(76) Inventors: James Kaput, Little Rock, AR (US); Nancy Fogg-Johnson, Villanova, PA (US)

Correspondence Address: BELL & ASSOCIATES, 58 West Portal Avenue No. 121, SAN FRANCISCO, CA, 94127, US

(21) Appl. No.: 12/312,897

(22) PCT Filed: Nov. 30, 2007
(86) PCT No.: PCT/US07/24715

§ 371 Date: Dec. 20, 2009

Related U.S. Application Data
(60) Provisional application No. 60/872,262, filed on Dec. 1, 2006.

(51) Int. Cl. A61B 5/00; G06Q 99/00; G06F 17/30

(52) U.S. Cl. 600/301; 705/1.1; 707/600; 707/E17.01

(57) ABSTRACT

This invention relates generally to providing nutrigenomic information tailored to that of the customer so that the customer can make informed decisions regarding diet, exercise, risks of disease and other health issues that result in a healthier lifestyle and prolonged lifespan. In particular the invention provides systems for research and commercial purposes, particularly for research for improving dietary constituents, personalized nutrition and diets, and of nutrient-gene interactions involved in diseases. The invention further relates to a method for doing business encompassing establishing and running a nutrigenomic research super-market and providing validated nutrient intake data to health care practitioners.

### RELATIONSHIP TO OTHER APPLICATIONS

The present application claims priority to and claims the benefit of U.S. Provisional Patent Application Ser. No. 60/872,262 entitled “Method For Determining Personalized Nutrition and Diet Using Nutrigenomics and Physiological Data”, filed Dec. 1, 2006, which is herein incorporated by reference in its entirety for all purposes.

### FIELD OF THE INVENTION

This invention relates generally to systems of monitoring and recording nutritional data associated with individual food items, systematically and quantitatively assessing nutrient intake in humans, and correlating that nutrient intake data with nutrigenomic, health, physiologic, other biological, and environmental data for research and commercial purposes, particularly for research into nutrient-gene interactions involved in chronic diseases, examples of which include type 2 diabetes mellitus (T2DM), obesity and metabolic syndrome. The invention further relates to a method for doing business encompassing establishing and running a nutrigenomic research supermarket and providing validated nutrient intake data to health care practitioners and their applications for clinical and consumer use.

### BACKGROUND

Health and susceptibility to disease result from an interaction among genes and environmental factors. Experimental evidence has proven that different genetic makeups respond differently to certain nutrients, and that dietary recommendations should differ among individuals (Ordovas, J.MaC. D., Gene-Environment Interactions: Defining the Playfield, in Nutritional Genomics. Discovering the Path to Personalized Nutrition, J. Kaput, Rodriguez, R. L., Editor. 2006, John Wiley and Sons: Hoboken, N.J. p. 57-76). The concept and study of nutrient—gene interactions is called nutrigenomics, a field of study that is beginning to provide a molecular understanding of the influence of environmental factors on an individual's health (Kaput, J. and Rodriguez, R. L. (2004) Nutritional genomics: the next frontier in the postgenomic era. Physiol. Genomics 16: 166-177). However, this field is in its infancy, and most of the nutrient—gene interactions responsible for health maintenance or disease progression have not been identified or carefully studied. While high throughput technologies are revolutionizing the study of DNA, RNA, proteins, and metabolites, the quantitative analysis of nutrient intake, a known contributor to development of chronic disease, remains a significant limitation in biomedical research. The explanation is straightforward: it is challenging to quantitatively assess nutrient intake in humans.

A recent article by Norman et al. (Am. J. Prey. Med. (2007) 33(4): 336-345) notes a number of trends. The rapid developments in interactive technologies in terms of processing power, data transmission, and data storage lead to a continuing evolution of eHealth interventions. Programs have evolved from first-generation technology that facilitated intervention tailoring with computers to generate printed materials. Second-generation eHealth interventions allow for direct interaction between the participant and the technology to increase capabilities beyond tailored feedback messages. Norman et al. stated that this second generation of interventions has allowed participants to select relevant psycho-educational information, report on goals and track their progress, and provide and receive social support either via bulletin boards or synchronous chat rooms. A third generation of eHealth technologies is already emerging. The interventions described by Norman et al. (2007, supra) consisted of desktop applications. However, mobile devices such as handheld computers, cellular telephones, and text messaging devices are emerging as new platforms for delivering health information. These platforms are also incorporating new functions such as sensing, monitoring, geospatial tracking, location based knowledge presentation, and a host of other information processes that will potentially enhance the ability for accurate assessment and tailored feedback. Research has already been conducted using PDA devices for ecologic momentary assessment (EMA), where real-time self-reported data are collected throughout a person's day. The EMA concept can be expanded to “ecologic momentary intervention,” such as “just-in-time” prompting for a behavior change based on some set of predefined conditions. eHealth behavior change interventions are still in the preliminary stages of development, and the potential for novel technologies to affect health behaviors is just beginning to be evaluated. While eHealth is progressing, it is clear that more research is needed to better determine how technology can be incorporated into programs to enhance behavior change outcomes. (See Norman et al., 2007, supra.)

In addition, a number of business models and commercial entities are currently providing personalised genetic and health information (eHealth technologies) for subscribers. These include Genomic Health, Inc. (Redwood City, Calif., USA), 23andMe, Inc. (Mountain View, Calif., USA), Navigenics, Inc. (Redwood Shores, Calif., USA), and DeCODE Genetics, Inc. (Reykjavik, Iceland).

Existing eHealth technologies have been shown in small studies to be effective in improving health, but most of the studies have not been done correctly (some have no eHealth controls to compare with the intervention group). One of the reasons is the difficulty in getting people to use the eHealth software systems largely due to the necessities of the individual to interact with the system in person and input queries to the databases (see Harmon, A., New York Times, Nov. 17, 2007).

Various relevant nutrigenomic methods are discussed in US patent publication No. 20040131658 entitled “Identification of Diet-regulated Disease-associated Genes” (U.S. Ser. No. 10/700,305, filed Oct. 31, 2003) and US patent publication No. 20050158734 entitled “Alleles Corresponding to Various Diet-Associated Phenotypes” Ser. No. 10/914,723, filed Aug. 9, 2004) both to James Kaput; the entire disclosure of this application is hereby incorporated by reference for all purposes.

### SUMMARY OF THE INVENTION

In one embodiment the invention provides a method for providing an individual with a personalized diet for said individual, the personalized diet optimised to reduce the likelihood that said individual will succumb to a disease or disorder, the method comprising the steps of (i) collecting nutritional information relating to one or more consumable items procured by the individual; (ii) storing the collected nutritional information in a first database; (iii) collecting biological information from the individual; (iv) storing the collected biological information in a second database or the first database; (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the method providing the individual with a personalized diet for said individual.

In another embodiment, the invention provides a system for providing an individual with a personalized diet for said individual, the personalized diet optimised to reduce the likelihood that said individual will succumb to a disease or disorder, the system comprising: a microprocessor, a first read-write digital storage device, and, in some embodiments, a second read-write digital storage device, a digital output device, wherein the microprocessor, the first read-write digital storage device, the second read-write digital storage device, and the digital output device are in electronic or photonic communication with one another, a software operating system, a software program comprising an algorithm that performs the steps of (i) collecting nutritional information relating to one or more consumable items procured by the individual; (ii) storing the collected nutritional information as a component of a first database in the first read-write digital storage device; (iii) collecting biological information from the individual; (iv) storing the collected biological information as a component of in a second database in the second read-write digital storage device; (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the system providing the individual with a personalized diet for said individual.

In another embodiment, the invention provides a nutritional protocol for determining the components of a personalized diet for an individual, the nutritional protocol comprising the steps of (i) collecting nutritional information relating to one or more consumable items procured by the individual; (ii) storing the collected nutritional information as a component of a first database in a first read-write digital storage device; (iii) collecting biological information from the individual; (iv) storing the collected biological information as a component of in a second database in a second read-write digital storage device (or in the first database); (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the nutritional protocol providing the individual with a personalized diet for said individual.

In another embodiment, the invention provides a clinical protocol for determining the components of a personalized diet for an individual, the clinical protocol comprising the steps of (i) collecting nutritional information relating to one or more consumable items procured by the individual; (ii) storing the collected nutritional information as a component of a first database in the first read-write digital storage device; (iii) collecting biological information from the individual; (iv) storing the collected biological information as a component of in a second database in a second read-write digital storage device (or in the first database); (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the clinical protocol providing the individual with a personalized diet for said individual.

In another embodiment, the invention provides a clinical protocol for determining an exercise regime for an individual, the clinical protocol comprising the steps of (i) collecting nutritional information relating to one or more consumable items procured by the individual; (ii) storing the collected nutritional information as a component of a first database in a first read-write digital storage device; (iii) collecting biological information from the individual; (iv) storing the collected biological information as a component of in a second database in a second read-write digital storage device (or in the first database); (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the clinical protocol providing the individual with an exercise regime for said individual.

In another embodiment, the invention provides a personalized diet, the personalized diet determined by performing the steps of (i) collecting nutritional information relating to one or more consumable items procured by the individual; (ii) storing the collected nutritional information as a component of a first database in s first read-write digital storage device; (iii) collecting biological information from the individual; (iv) storing the collected biological information as a component of in a second database in s second read-write digital storage device (or in the first database); (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); thereby determining a personalized diet for said individual.

In another embodiment, the invention provides a research supermarket for determining personalized diets, the research supermarket comprising: a microprocessor, a first read-write digital storage device, a second read-write digital storage device, a digital output device, wherein the microprocessor, the first read-write digital storage device, the second read-write digital storage device, and the digital output device are in electronic or photonic communication with one another, a software operating system, a software program comprising an algorithm that performs the steps of (i) collecting nutritional information relating to one or more consumable items procured by the individual; (ii) storing the collected nutritional information as a component of a first database in the first read-write digital storage device; (iii) collecting biological information from the individual; (iv) storing the collected biological information as a component of in a second database in the second read-write digital storage device (or in the first database); (v) repeating steps (i) through (iv) at predetermined intervals; (vi) at any one interval, comparing the collected nutritional information with the collected biological information; (vii) correlating the collected nutritional information with the collected biological information, thereby generating a statistical correlation that associates nutrient, health, and biological information with health or disease outcomes; (viii) providing the individual with a personalized diet optimised to reduce the likelihood of the individual succumbing to a disease or disorder; (ix) repeating any of steps (i) through (viii); the system providing the individual with a personalized diet for said individual.

In another embodiment, the invention provides a method for collecting and correlating at least two datasets, wherein each dataset comprises at least one element in common, wherein a first dataset includes information relating to one or more consumable items purchased by an individual, and wherein a second dataset includes information relating to physiological data or genetic parameters of an individual; the method comprising: (1) establishing a facility comprising a first database where unique product identifiers are associated with food items and are used to track and record data associated with such food items is the first database; (2) tracking and recording data associated with food item purchases by an individual and storing such data in a second database; (3) measuring and recording physiological or genetic parameters of an individual, either before and/or after purchase of food items and storing such data in a third database; and (3) correlating food item purchase data from the second database with data related to changes in physiological or genetic parameters of the individual from the third database.

In another embodiment, the invention provides a method for collecting and correlating at least two sets of related data, wherein a first set of data includes information relating to one or more consumable items purchased by an individual, and wherein a second set of data includes information relating to physiological data or genetic parameters of an individual; the method comprising: (1) establishing a facility where unique product identifiers are associated with food items and are used to track and record data associated with such food items; (2) tracking and recording data associated with food item purchases by individuals and storing such data in a database; (3) measuring and recording physiological or genetic parameters of an individual, either before and/or after purchase of food items and storing such data in a database; and (3) correlating food item purchase data with data related to changes in physiological or genetic parameters of the individuals.

In a preferred embodiment the method, system, protocol, personalized diet, or research supermarket further comprises measuring and recording food item consumption. In another preferred embodiment the physiological data comprises epigenetic data, genetic data, genomic data, and nutrigenomic data. In a further preferred embodiment, the physiological data comprises measurements of heart rate, breathing rate and volume and blood pressure. In a still further preferred embodiment, the physiological data comprises measurements of weight, BMI, HDL, LDL, cholesterol, glucose, lipids, HbA1c, blood pressure, biomarkers of inflammation, TNF-α, HsCRP, leukotrienes, prostaglandins, and hormones. In a more preferred embodiment, the hormones comprise insulin, glucagon, and leptins. In another preferred embodiment the physiological data is collected before and after consumption of a food item. In a yet further preferred embodiment the disease or disorder is selected from the group consisting of type 2 diabetes. mellitus (T2DM), obesity, metabolic syndrome, Alzheimer's disease, cardiovascular disease, and cancer. In a most preferred embodiment, the disease or disorder is type 2 diabetes mellitus.

In another preferred embodiment the genetic data comprises a named gene associated with a disease or disorder. In a more preferred embodiment the named gene predisposes an individual to a disease or disorder. In one more preferred embodiment the named gene is selected from the group of named genes of Table 1.

In another preferred embodiment the genetic data comprises a named single nucleotide polymorphism (SNP) associated with a disease or disorder. In a more preferred embodiment the named SNP predisposes an individual to a disease or disorder.

It should be noted that in the present disclosure, a second database and a second read-write digital storage device are referred to for convenience in order to show separate storage of data, but it is understood that any database or storage device may be used that may be the same as of physically separate from any other database or storage device. Also, when a food item is referred to, it is understood that multiple food items may likewise be used.

### BRIEF DESCRIPTION OF THE FIGURE

FIG. 1 illustrates a table showing different types of standard bar code and the amount of information that can be stored in each.

### DEFINITIONS

The term “Food” as used herein refers to any nutrient and any biologically active substance without a defined nutritional role taken in to the body that is subsequently metabolized, incorporated and processed by the body; as such, foods also include bioactives such as but not limited to dietary supplements such as curcumin or ginketin, which may not be metabolized, but which may alter expression of genetic information through known or unknown molecular mechanisms; foods include solid and liquid foods, vitamins, minerals, juices, and water.

The term “Purchase” as used herein refers to the acquiring of an article whether or not payment is made.

The term “Consume” as used herein means eating, drinking or placing into the body by any means, including by application to the skin.

The term “Supermarket” as used herein refers to any market, store, shop, or facility, including restaurants, from which foods may be obtained or procured.

The term “Facility” is used to describe any establishment, whether physical or virtual, from where food items may be ordered or obtained.

The term “Unique product identifier” as used herein refers to any identification device such as a tag, label, bar code, RFID tag and the like, that may be physically associated with an item and that may retain or convey information about that item. The tag does not have to be absolutely unique, only unique enough to practically provide identification for the item being identified.

The term “Biological information” as used herein refers to any information, analysis, measurement, data, statistical correlation, compound, composition, element, etc., that is obtained from a biological source, information such as, but not limited to, such as physiological information, pharmacological information, pharmacokinetic information, biochemical information, immunological information, endocrinological information, genetic information, genomic information, SNP information, and epigenetic information.

### General Representations Concerning the Disclosure

In the present disclosure reference is made to particular features of the invention. It is to be understood that the disclosure of the invention in this specification includes all appropriate combinations of such particular features. For example, where a particular feature is disclosed in the context of a particular embodiment or a particular claim, that feature can also be used, to the extent appropriate, in the context of other particular embodiments and claims, and in the invention generally.

The embodiments disclosed in this document are illustrative and exemplary and are not meant to limit the invention. Other embodiments can be utilized and structural changes can be made without departing from the scope of the claims of the present invention. In the present disclosure, reference is made to particular features (including for example components, ingredients, elements, devices, apparatus, systems, groups, ranges, method steps, test results, etc). It is to be understood that the disclosure of the invention in this specification includes all possible combinations of such particular features.

As used herein and in the appended claims, the singular forms “a”, “an”, and “the” include plural reference unless the context clearly dictates otherwise. Thus, for example, a reference to “a part” includes a plurality of such parts, and so forth.

The term “comprises” and grammatical equivalents thereof are used herein to mean that, in addition to the features specifically identified, other features are optionally present. For example, a composition “ comprising” (or “which comprises”) ingredients A, B and C can contain only ingredients A, B and C, or can contain not only ingredients A, B and C but also one or more other ingredients. The term “consisting essentially of” and grammatical equivalents thereof is used herein to mean that, in addition to the features specifically identified, other features may be present which do not materially alter the claimed invention. The term “at least” followed by a number is used herein to denote the start of a range beginning with that number (which may be a range having an upper limit or no upper limit, depending on the variable being defined). For example “at least 1” means 1 or more than 1, and “at least 80%” means 80% or more than 80%. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)—(a second number)”, this means a range whose lower limit is the first number and whose upper limit is the second number.

Where reference is made herein to a method comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility). Where reference is made herein to “first” and “second” features, this is generally done for identification purposes; unless the context requires otherwise, the first and second features can be the same or different, and reference to a first feature does not mean that a second feature is necessarily present (though it may be present). Where reference is made herein to “a” or “an” feature, this includes the possibility that there are two or more such features (except where the context excludes that possibility). For example, a composition which comprises a protein and a micronutrient, the composition can comprise two or more proteins and/or two or more micronutrients. Where reference is made herein to two or more features, this includes the possibility that the two or more features are replaced by a lesser number or greater number of features providing the same function (except where the context excludes that possibility). The numbers given herein should be construed with the latitude appropriate to their context and expression; for example, each number is subject to variation which depends on the accuracy with which it can be measured by methods conventionally used by those skilled in the art.

This specification incorporates by reference all documents referred to herein and all documents filed concurrently with this specification or filed previously in connection with this application, including but not limited to such documents that are open to public inspection with this specification.

### DESCRIPTION OF THE INVENTION

The present disclosure encompasses and describes a novel method to systematically and quantitatively assess nutrient intake in humans and to correlate that nutrient intake with nutrigenomic data for research purposes (including commercial research and analysis), particularly for research into nutrient-gene interactions involved in chronic diseases, using type 2 diabetes mellitus (T2DM) as an example. The disclosure additionally encompasses a business method for conducting a business comprising establishing and running a research supermarket and gathering and providing validated nutrient intake data to health care practitioners.

The method comprises establishing a research supermarket where unique product identifiers are used to track and record data associated with food purchases and wherein such methods can be used to track nutrient intakes for an individual or family. Data about which food items are consumed are converted through the use of food composition databases to component nutrients and amounts consumed, and these data are correlated with nutrigenomic data for research purposes.

Food intake is further monitored by requiring study participants (also referred to a; patients, customers or consumers) to return unused, unconsumed food, although other methods of monitoring uneaten foods are not excluded. Nutrient intakes monitored by this approach are then analyzed with respect to (and with analysis of) clinical, genetic, and nutrigenomic data. These clinical, genetic, and nutrigenomic data are obtained by high throughput analyses of DNA, RNA, proteins, and metabolites, some of which are identical to data obtained from clinical tests (for example, cholesterol levels, glucose levels, lipid levels, and the like) as well as measurements of liver and other tissue activity (for example, using absorption, distribution, metabolism, and excretion (ADME) assays).

Food “purchases” are monitored using unique product identifiers associated with individual food items linked to a database encoded in or accessible via use of “smart” cards, similar in function to “club cards” used by commercial enterprises.

In certain embodiments, the product identifier may be a bar code or an RFID tag physically associated with a food item

Product identifiers may store various types and amounts of information. Information may include the type and weight of a particular food article, or may include nutritional information such as calorific content, types of nutrients, such as fat, protein, fibre, carbohydrates, minerals, vitamins, and other bioactive substances, and the like. The product identifiers can be labels, barcodes, or any other machine-readable code system. Barcodes may also contain nutrigenomic data—that is, data describing gene—nutrient interactions for the particular product that can be matched to a consumer's nutrigenomic (nutrients optimal for their genetic makeup) requirements.

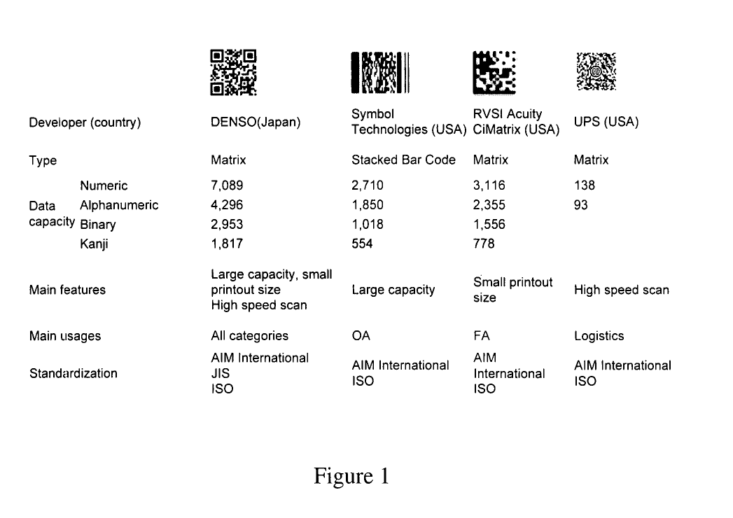

Bar codes, particularly two-dimensional ones (that contain information in both the vertical and horizontal directions) can store a sizeable amount of information about a particular food item. FIG. 1 illustrates a table showing different types of standard bar code and the amount of information that can be stored in each.

The bar code may be applied to the food article and read by means of a reader functionally linked to a computer, which computer is functionally linked to a central processor and a data storage device. The computer may be further linked to a network of computers such as a local area network or the World Wide Web. Exemplary barcodes are shown in FIG. 1.

In another embodiment, product identifier may be a memory storage means such as an RFID chip. Newer 13.65 MHz RFID chips can store about 2000 bits of data or more, such that extensive nutritional and biochemical information may be stored and associated with a food item.

Data from bar codes or RFID tags and the like, may be captured in various ways, for example by reading the bar codes of RFID tags using a laser scanner or radio frequency reader at the supermarket check-out, or by monitoring and recording food-associated data ordered on-line using a simple relational database. Data is stored in a memory means and retrieved when needed via computer for processing and analysis.

In another embodiment, food is ordered online (or by any other remote method such as by fax, or phone) and delivered to the consumer.

In another embodiment, food purchases are obtained from existing or new supermarkets, which regularly track purchases with barcodes. In this embodiment, the barcodes are for the product, and nutrient content can be linked to the product from manufacturers. Purchases in restaurants and other food stores are obtained similarly through credit card receipts of club cards. After obtaining individual and family consent, data from purchases is done through electronic transfer.

In an alternative embodiment, bar codes, RFID tags or other product identifiers and individual's “smart cards” may be programmed to guide consumers to optimal or recommended selections of food products according to the consumer's genetic makeup and composition of food products. Food choices can be “directed” through use of a smart card or personal data assistant, cell phone, or other communication device. This device will be coded for an individual's or family's recommended nutrient intake and keyed to nutrient composition on smart tags for on-shelf food products in the research supermarket. These cards will be programmed with optimum calories, macro-, and micro-nutrient intakes based eventually on genetic, clinical, lifestyle, age, health status, and their interactions. Products must be previously labelled with RFID technology. Alternatively, each device has a barcode linked to algorithms, which correlate nutrient content to an individual's nutrient requirements.

### Information Content

A number of different parameters may comprise and/or describe the information that is used to provide a personalized nutrition or personalized diet, including, for example, but not limited to, nutritional information from food, nutritional information from other diets, nutritional information from supplement, biological information from the individual, biological information from a peer, biological information from a peer group, biological information from a peer population, biological information from a general population, biological information from a non-peer group or population, biologic al information from experimental animals, biological information from experimental cell cultures, and the like. The biological information can be physiological measurements, such as, but not limited to, body mass index (BMI), heart rate, heart volume, heart output, blood pressure (both systolic, diastolic, and the like), blood flow, blood enzymes, blood pH, breathing rate, lung capacity, oxygen uptake, carbon dioxide removal, muscle strength, liver enzymes, kidney excretion rates, levels of circulating metabolites and carrier proteins, such as but not limited to, HDL, LDL, cholesterol, glucose, insulin, lipids, HbA1c, biomarkers of inflammation, TNF-α, leukotrienes, prostaglandins, and hormones; and quality of life assessments (for example, six minute walk distance, oxygen delivery using cycle ergometry, and the St. George's Respiratory Questionnaire (SGRQ), and the like. The biological information can be metabolic information, such as but not limited to, samples taken from tissue, such as liver, muscle, lung, blood, prostate, breast, pancreas, gut, stomach, heart, cerebrospinal fluid, urine, lymph nodes, lymph, and the like.

Those of skill in the art will know that there are many different methods used to mine data from a database or a dataset, including, but not limited to, algorithms that employ key word searches, algorithms that employ full text searches, and algorithms that analyse statistical significance between single, or groups of, candidate data or information.

### Analyses of Information

Many methods of statistical analysis may be used to correlate the collected nutritional information with the collected biological information, thereby generating a statistical correlation that associates nutrient, health, and biological information with health or disease outcomes. Such statistical methods are well know to those of skill in the art and can compare single parameters or measurements or can compare multiple parameters or measurements. An exemplary method is disclosed herein, but other methods can and may be used with the invention.

### Coefficient of Correlation

The coefficient of correlation indicates an association between two variables.

The formula is:

r_{xy}=COV(X,Y)/stdₓ·std_{y}

where
r_{xy} is the correlation between X and Y
COV(X,Y) the covariance between X and Y
stdₓ is the standard deviation of X
std_{y} is the standard deviation of Y.

### Coefficient of Determination

Symbolized as r2, the coefficient of determination is the square of the correlation coefficient. The coefficient of determination can have only positive values ranging from r2=+1.0 for a perfect correlation (positive or negative) down to r2=0.0 for a complete absence of correlation. For example, if the coefficient of determination between X and Y is 0.55 (55%), the correlation coefficient is either +0.74 or −0.74, then 55% of the variability in X is explained by a variability in Y.

### Statistical Significance

Statistical significance may be used to test if the result (here the coefficient of correlation) gives a high degree of confidence that there is a relationship between X and Y.

To test if r is significantly different than 0 by building an interval of confidence around r, the following may be performed. If 0 is included in the interval, r will not be considered as significantly different than 0 and if 0 is not included in the interval, r will be considered as significantly different than 0.

To build that interval, a Fisher's Z-transformation may be used (1.96 in the above formula is at the 95% confidence level).

The formula is:

1/2 ln(1+r/1−r)±1.96·√1/n−3=1/2 ln(1+ρ/1−ρ)
with
n the sample size.
r the correlation coefficient.
ρ the high end and low end case of the interval.

If 0 is not between the 2ρ, r is significantly different than 0.

In certain embodiments, the methods include the conducting of genetic or physiological tests, or the use of genetic or physiological data, separately collected, in conjunction with data gathered from food purchase and consumption tracking.

Genetic and physiological data may be collected by patient survey or by direct genetic or physiological analysis. Genetic testing may focus on specific genes and alleles of genes associated with various disease states, such as diabetes and obesity. For example, diet-regulated disease-associated polynucleotides identified by the methods described in the inventor's applications U.S. Ser. Nos. 10/700,305 and 10/914,723, may be quantified for an individual or population and correlated with nutritional intake information. Genetic data may also be separately obtained through use of whole genome arrays analyzing single nucleotide polymorphism, copy number variants, or any other genetic variation (such as epigenetic) and through use of technologies to assess all genetic variations. Epigenetic variation is particularly important as it relates to metabolic and nutrient-induced changes in gene expression. Included in these analyses will be assessments of an individual's genetic ancestry, which is known to affect gene—nutrient and gene—disease associations.

An exemplary list of gene names wherein a gene comprising a polynucleotide has been associated with a particular disease, condition, or disorder, are disclosed below and in Table 1.

TABLE 1
Possible Candidate Genes for Type 2 Diabetes^{a}

| Mutated | | | |
|---|---|---|---|
| Gene | Function | Effect | Linked to |
| HNF-4α, HNF-1β | Transcription Factors | ↓Insulin Secretion | MODY (maturity-onset |
| IPF-1, NeuroD1 | | | diabetes in the young) |
| | | | human |
| HNF-1α, | Transcription Factor | ↓Insulin Secretion | MODY Oji-Cree diabetes |
| Glucokinase | Glucose Metabolism | ↓Insulin Secretion | MODY |
| Calpain-10 | Protease | Unknown | Diabetes 2 in Mexican |
| | | | and African Americans |
| PPARγ | Transcription Factor | ↓Insulin Sensitivity | Diabetes 2 |
| Insulin | Transmits Insulin Signals | ↓Insulin Secretion | Human diabetes (rare) |
| Receptor | into cells | and Sensitivity | mouse model |
| IRS1 and -2 | Insulin Signaling | ↓Insulin Sensitivity | Mouse Models |
| Akt2 | Insulin Signaling | ↓Insulin Sensitivity | Mouse Models |
| 11-β-HSD | Glucocorticoid Synthesis | ↑Blood Lipid, | Mouse Models |
| | | ↓Insulin Secretion | |
| UCP2 | ↓ATP Synthesis | ↓Insulin Secretion | Mouse Models |
| Resistin | Fat Cell “hormone” | ↓Insulin Sensitivity | Mouse Studies |
| Adiponectin | Fat Cell “hormone” | ↑Insulin Sensitivity | Mouse, Human Studies |

^{a}Adapted from: Marx, J., Unraveling the causes of diabetes. Science, (2002) 296: p. 686-689.

Aes, amino-terminal enhancer of split. Aes is a co-repressor of NFκβ, which is activated in insulin resistant tissues. Aes is less abundant in muscle tissue of humans with a family history of T2DM relative to those with no family history.

Mark4, MAP/microtubule affinity-regulating kinase. Mark4 participates in the Wnt/β-catenin signaling pathway, which, when misregulated, may result in cancer. GSK-3β (glycogen synthase kinase 3β) is a negative regulator of this pathway and is downregulated when cells are exposed to Wnts. GSK-3 is also a suppressor of glycogen synthase and insulin receptor substrate 1. Pdtgs, Prostaglandin D Synthase synthesizes prostaglandin G2 that is a precursor to prostaglandin J₂ (PGJ₂). PGJ₂, is in turn, a precursor to 15-deoxy-Δ¹²⁻¹⁴-PGJ2, the primary ligand of the peroxisome proliferator activated receptor (PPAR-γ).

Pparbp, Peroxisome proliferator activated receptor binding protein. Pparbp is a coactivator of PPAR-α, -γ, retinoic acid receptor-α (RAR-α), retinoic X receptor (RXR), estrogen receptor (ER), and thyroid receptor β1 (TR-β-1). PPAR-γ appears to be one of the key regulators of glucose and lipid homeostasis.

Fabp, fatty acid binding protein. Fabp may function by targeting its ligand to the nucleus and may participate in regulation of gene expression by binding to PPAR-γ.

Testing may include qualitative and quantitative measurement of the presence or expression of any polynucleotide or protein, such as DNA, RNA, enzymes or and proteins. Metabolites also may be measured such as any of the metabolic intermediates of glycolysis or Kerb's cycle or of any known metabolic process.

Physiological measurements of weight, BMI, HDL, LDL, cholesterol, glucose, HbA1c, blood pressure, biomarkers of inflammation, such as TNF-alpha, HsCRP, leukotrienes, or prostaglandins, or any morphological, physiological, or lifestyle (activity levels including exercise) and the like, may be correlated with nutritional consumption data for an individual or group of individuals. Additionally, hormone levels such as insulin, glucagons, leptin levels may be measured at various intervals and correlated with consumption data. Such physiological data may be collected on a schedule linked with the schedule of consumption, for example at particular times before and after consumption of a meal.

Correlation and statistical significance between different data can be measured as disclosed above. Meaningful correlates are known to those of skill in the art. For example, a meaningful correlate can result in a coefficient of correlation of 0.7<r<1.0; 0.49<r2<1.0. For example, r can be 0.7, 0.75, 0.8, 0.85, 0.9, 0.95, 0.96, 0.97, 0.98, 0.99, and 1.0, and any value therebetween.

One advantage of this novel research supermarket facility is that a database of all the foods and their nutrient composition will be established, and cross-linked to particular individuals, so that a more accurate record of the nutrient composition of diet is obtained for consumers or individual study participants. It is well known that most dietary assessment tools are inaccurate which reduces the significance and reliability of gene—disease associations, gene—nutrient interactions that are the basis of health and disease processes, and genetic tests for diagnosis and prognosis (see Cade, J. et al. (2002) Development, validation and utilisation of food-frequency questionnaires—a review. Public Health Nutr. 5: 567-587; Dennis, L. K. et al. (2003) Developing a scoring method for evaluating dietary methodology in reviews of epidemiologic studies. J. Am. Diet. Assoc. 103: 483-487).

In various embodiments, the research supermarket is established for an extended period of time, and data is consistently collected for a period of, for example, at least 3 months, at least 6 months, at least 12 months, at least 24 months, at least 36 months, or at least 48 months, or aperiod of time determined to be necessary to evaluate effects of nutrients on genetic expression of health or disease.

The business model described herein is unique in that an operator establishes and operates the physical or virtual supermarket as a part of its own research enterprise, or alternatively, on a contractual basis for others. The combination of research supermarket coupled with nutrigenomic analyses of the response of individuals or groups to differing food intakes represents a novel business concept unique to the inventors. No facility exists to effectively and efficiently evaluate nutrient intake in a free-choice environment or in a situation where individuals are given dietary advice and/or products, such as via home delivery, and allowed to implement it themselves.

An extension of this business model is that nutrient intakes can be assessed through tracking of purchases at existing or new retail supermarkets. These data are usually used only for marketing, product sales, and re-stocking. Providing individualized nutrient-directed purchases to individuals, health care practitioners, including but not limited to family and specialty physicians, nutritionists, genetic counsellors, registered or licensed dieticians, will aid in maintaining health and improve the diagnosis, treatment, and outcomes for diseases.

In a related embodiment an individual person carries a card (or some form of media substrate) with, for example, a read/write magnetic strip or chip, that can store information about food purchases and communicate that information to a central database to be analyzed and correlated with genomic, physiological and disease data for the individual.

Chronic diseases, including type 2 diabetes mellitus, obesity, metabolic syndrome, Alzheimer's, cardiovascular diseases, and certain cancers (among others), are generally produced by the interplay of environmental factors and genetic mechanisms.

Assays to measure physiological and metabolic values are well known to those of skill in the art. For example, electrocardiograms, blood flow, blood pressure analysis, renal flow, neurological activity, lung capacity, lung permeability, muscle strength, and the like are routinely performed by those of skill in the art. For example, measurements of blood components, such as Hb, ppO2, acidity, glucose, insulin; liver enzymes, such as cytochrome P450 molecules and other detoxification enzymes; ureanalysis, and the like are routinely performed by those of skill in the art.

There are many methods well known to those of skill in the art that can be used to identify polymorphisms, mutations, and methylation of chromatin (see for example, U.S. Pat. No. 7,247,428. In one aspect, epigenetic changes, such as the methylation state of chromatin, can inform as to the likelihood that a gene or group of genes may be active.

There are various art-recognized assays for assessing the methylation state at particular CpG sequences, once the sequence region comprising them has been identified so that specific primers and/or probes can be constructed. Such assays include: DNA sequencing methods; Southern blotting methods; METHYLIGHT. (fluorescence-based real-time PCR technique described by Eads et al., Cancer Res. 59:2302-2306, 1999; U.S. Pat. No. 6,331,393); MS-SNuPE (Methylation-sensitive Single Nucleotide Primer Extension assay described by Gonzalgo & Jones, Nucleic Acids Res. 25:2529-2531, 1997; U.S. Pat. No. 6,251,594); MSP (Methylation-specific PCR assay described by Herman et al. Proc. Natl. Acad. Sci. USA 93:9821-9826, 1996; U.S. Pat. No. 5,786,146); and COBRA (Combined Bisulfite Restriction Analysis methylation assay described by Xiong & Laird, Nucleic Acids Res. 25:2532-2534, 1997). Such methylation assays are used, for example, to analyze genomic DNA sequence regions that exhibit altered methylation patterns (hypermethylation or hypomethylation) in cancer patients. These methylation-altered DNA sequences are, in turn, useful in indirect therapeutic applications as diagnostic, prognostic and therapeutic markers for human cancer.

Assays for the discovery of novel differentially methylated CpG sequences are less numerous in the art, but include such methods as: restriction landmark genomic scanning (RLGS; Eng et al., Nature Genetics 25:101-102, 2000; Costello et al., Nature Genetics 25:132-138, 2000; Zhu et al., Proc. Natl. Acad. Sci. USA 96:8058-8063, 1999); methylated CpG island amplification (MCA; Toyota et al., Cancer Res. 59:2307-2312, 1999; WO 00/26401A1), differential methylation hybridization (DMH; Yan et al., Clin. Canc. Res. 6:1432-1438, 2000); arbitrarily primed-polymerase chain reaction (AP-PCR; Liang et al., Genomics 53:260-268, 1998); and RLGS in combination with virtual genome scans (VGS; Rouillard et al., Genome Research 11:1453-1459, 2001) derived from the sequence of the human genome to predict sequence of RLGS fragments (spots).

The present invention provides systems that integrate nutritional information, genetic information, physiological information, and metabolic information, diets, and methods for automatically generating information and data that can be regularly forwarded to an individual thereby avoiding the complications associated with the current art since it delivers such information with little effort on the part of the individual or consumer.

### Examples

The invention will be more readily understood by reference to the following examples, which are included merely for purposes of illustration of certain aspects and embodiments of the present invention and not as limitations.

### Example I

### Nutrigenomics Supermarket for Providing Personalized Nutrition

In one exemplary embodiment, a supermarket is established in which a variety of food items are stocked. Each item is tagged with a unique product identifier code (UPC). Each UPC encodes (or contains a code that allows retrieval of) information relating to the nutritional and/or biochemical composition of the item. A reader system is provided to read the UPC upon purchase or acquisition of an item by a subject. A relational database encoded on a computer is provided. Data about item purchases is associated in the database with the individual who made the purchase. In certain embodiments, an accounting is made of exactly what food items have been consumed, not just purchased. This may be facilitated by use of a food diary, in any format (for example, paper, electronic, and the like). Physiological and/or genetic data such as relating to allele number or expression or relating to physiology such as but not limited to weight, BMI, HDL, LDL, cholesterol, glucose, blood pressure and the like, is or has been collected for an individual. In certain embodiments, physiological data is collected at regular intervals and at various times before and after consumption of a meal. These data are stored in a database and is correlated with item purchase information. Statistical methods are used to determine statistically valid patterns and relationships between nutritional consumption and genetic and physiological measurements for individuals and populations. In one published study, significant gene-diet interactions were found between the −1131T>C polymorphism in the APOA5 gene and polyunsaturated fatty acid (PUFA) intakes. These interactions contributed to fasting serum triglyceride levels, and two markers of cardiovascular health, remnant like particles and lipoprotein particle size. These interactions were not found for the other variant of APOA5, (56C>G polymorphism). Specifically, fasting triglycerides and remnant-like particles (which are associated with cardiovascular disease) increased in subjects consuming polyunsaturated fats at greater than 6% of total energy only in individuals with the APOA5-1131C allele In addition, these effects were specific for n-6 fatty acids as opposed to n-3 fatty acids (also called omega 3 FA which are found in fish and fish oils as well as some microorganisms). These data suggest that n-6 PUFA-rich diets are related to a more atherogenic lipid profile in the subjects in the study. (See Lai, C. Q. et al. (2006) Dietary intake of n-6 fatty acids modulates effect of apolipoprotein A5 gene on plasma fasting triglycerides, remnant lipoprotein concentrations, and lipoprotein particle size: the Framingham Heart Study. Circulation 113: 2062-2070.)

Optionally, exercise and calorie expenditure for individuals is measured. This may be done by using an exercise diary or by measuring, constantly or intermittently, physiological parameters associated with exercise, such as heart rate, breathing rate and volume and blood pressure. Such data may additionally be stored in a database and correlated with nutritional intake data.

In one example, individuals in a clinical study of type 2 diabetes mellitus will purchase food at a research supermarket. Food purchases will be converted to nutrients. Each week, the individual will provide to the study all unused food which will be weighed and nutrient content calculated. These data will be uploaded to a relational database. Other datasets will include variants of candidate genes, whole genome SNP, gene copy variations, or epigenetic data, which also will provide information on ancestral backgrounds, and metabolite data obtained from clinical or research laboratories. If the study requires, activity levels will also be measured through questionnaires or activity monitors (for example, a recording heart rate monitor worn for extended periods of time). These data will be analyzed using dimensionality reduction algorithms capable of finding nonlinear functional relationships. Family members of participants who are predisposed to type 2 diabetes, or other chronic disease, but are not yet showing signs of the disease will also benefit from the research supermarket through being guided to select foods whose composition may be associated with delay or prevention of disease onset or mitigation of disease severity.

In another example of data obtained from existing supermarkets, data obtained from food purchases from supermarket databases will be obtained and converted to nutrient levels. These data can be provided to a health care practitioner, one example being a family physician, who can then incorporate nutrient purchases with physiological or clinical data. In an embellishment of this option, gene variant and or whole genome data can be obtained and provided to the health practitioner separately (as stand-alone genetic information) or converted to nutrigenomic (including clinical, metabolite, protein, and data from other low or high throughput analytic methods) to provide enriched information. Such information can be used to refine medical treatments, prescribe altered lifestyles including nutrient intake and activity levels, and monitor health or disease progression.

### Example II

### Identification of Epigenetic Changes

### Methylation Detection

In another example, the method of the present invention can be used to detect whether methylation has occurred in a sample, such as single known gene or multiple genes. The methylation detection method preferably uses genomic DNA to assay the degree of methylation.

The methylation detection method comprises a chemical or enzymatic approach for methylation-sensitive treatment of genomic DNA. Chemical treatments include the incubation of genomic DNA with sodium bisulfite, which selectively and completely converts non-methylated cytosines to uracils. The DNA is first heat-denatured and then treated with 5M bisulfite, pH 5-7. Following this, uracil glycosylase is used to convert uracils to abasic sites, which are then converted to 5′P-containing strand breaks via heat or alkali, as described above. Pretreatment of genomic DNA to remove pre-existing uracils is used prior to bisulfite treatment. This pretreatment consists of uracil glycosylase treatment in the presence of 5 mM hyclroxylamine, pH 7.

Enzymatic approaches that can be used to convert methylated cytosines to 5′P include treating genomic DNA with 5-methyl-cytosine glycosylase, which removes the methylated cytosines, leaving an abasic site, which is then converted to 5′P-containing strand break as described. Another enzymatic approach is to use methylation-sensitive restriction endonucleases, which only cut non-methylated sequences, producing directly 2 5′P—one on each strand.

A second strand directly in the genomic DNA sequence of interest is then synthesized. The primers used to synthesize the second strand must be specific, to avoid priming of other genomic regions. If bisulfite is used as a treatment, the actual sequence which is targeted by the primer will have changed since cytosines become uracils. Therefore the design of the primer must take this into account for adequate priming of the targeted region, i.e. guanines must become adenines in the primer design. Following this, a linker is ligated to the unique 5′P ends, and then PCR takes place.

The result of the PCR is a product whose amount is proportional to the degree of methylation (or non-methylation, depending on the method selected in Step 1) of the target sequence. The product consists of a range of sizes of DNA, starting at the primer and finishing at the positions of cutting, i.e. the positions of methylation (or non-methylation). This product is run on a sequencing gel to identify the positions that were cut at a nucleotide resolution.

The method for detecting methylation can also be applied to screen many genes simultaneously, such as a whole genome.

### Example III

### Identification of Gene Expression Changes

Isolation and Labeling of Sample cDNAs

Cells are harvested and lysed in 1 ml of TRIZOL reagent (5×10⁶ cells/ml; Life Technologies). In the alternative, tissue is homogenized and lysed in 1 ml of TRIZOL reagent (1 g tissue/10 ml). The lysates are vortexed thoroughly and incubated at room temperature for 2-3 minutes and extracted with 0.5 ml chloroform. The extract is mixed, incubated at room temperature for 5 minutes, and centrifuged at 15,000 rpm for 15 minutes at 4° C. The aqueous layer is collected and an equal volume of isopropanol is added. Samples are mixed, incubated at room temperature for 10 minutes, and centrifuged at 15,000 rpm for 20 minutes at 4° C. The supernatant is removed and the RNA pellet is washed with 1 ml of 70% ethanol, centrifuged at 15,000 rpm at 4° C., and resuspended in RNase-free water. The concentration of the RNA is determined by measuring the optical density at 260 nm.

Poly(A) RNA is prepared using an OLIGOTEX mRNA kit (QIAGEN) with the following modifications: OLIGOTEX beads are washed in tubes instead of on spin columns, resuspended in elution buffer, and then loaded onto spin columns to recover mRNA. To obtain maximum yield, the mRNA is eluted twice.

Each poly(A) RNA sample is reverse transcribed using MMLV reverse-transcriptase, 0.05 μg/μl oligo-d(T) primer (21 mer), 1×first strand buffer, 0.03 units/μl RNase inhibitor, 500 μM dATP, 500 μM dGTP, 500 μM dTTP, 40 μM dCTP, and 40 μM either dCTP-Cy3 or dCTP-Cy5 (APB). The reverse transcription reaction is performed in a 25 ml volume containing 200 ng poly(A) RNA using the GEM BRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) are synthesized by in vitro transcription from non-coding yeast genomic DNA. As quantitative controls, control InRNAs (YCFR06, YCFR45, YCFR67, and YCFR85) at 0.002 ng, 0.02 ng, 0.2 ng, and 2 ng are diluted into reverse transcription reaction at ratios of 1:100,000, 1:10,000, 1:1000, 1:100 (w/w) to sample mRNA, respectively. To sample differential expression patterns, control mRNAs (YCFR43, YCFR22, YCFR23, YCFR25, YCFR44, YCFR26) are diluted into reverse transcription reaction at ratios of 1:3, 3:1, 1:10, 10:1, 1:25, 25:1 (w/w) to sample mRNA. Reactions are incubated at 37° C. for 2 hr, treated with 2.5 ml of 0.5M sodium hydroxide, and incubated for 20 minutes at 85° C. to the stop the reaction and degrade the RNA.

cDNAs are purified using two successive CHROMA SPIN 30 gel filtration spin columns (Clontech). Cy3- and Cy5-labeled reaction samples are combined as described below and ethanol precipitated using 1 ml of glycogen (1 mg/ml), 60 ml sodium acetate, and 300 ml of 100% ethanol. The cDNAs are then dried to completion using a SpeedVAC system (Savant Instruments, Holbrook N.Y.) and resuspended in 14 μl 5×SSC/0.2% SDS.

Hybridization reactions contained 9 μl of sample mixture containing 0.2 μg each of Cy3 and Cy5 labeled cDNA synthesis products in 5×SSC, 0.2% SDS hybridization buffer. The mixture is heated to 65° C. for 5 minutes and is aliquoted onto the microarray surface and covered with an 1.8 cm² coverslip. The microarrays are transferred to a waterproof chamber having a cavity just slightly larger than a microscope slide. The chamber is kept at 100% humidity internally by the addition of 140 μl of 5×SSC in a corner of the chamber. The chamber containing the microarrays is incubated for about 6.5 hours at 60° C. The microarrays are washed for 10 min at 45° C. in low stringency wash buffer (1×SSC, 0.1% SDS), three times for 10 minutes each at 45° C. in high stringency wash buffer (0.1×SSC), and dried.

Reporter-labeled hybridization complexes are detected with a microscope equipped with an lnnova 70 mixed gas 10 W laser (Coherent, Santa Clara Calif.) capable of generating spectral lines at 488 nm for excitation of Cy3 and at 632 nm for excitation of Cy5. The excitation laser light is focused on the microarray using a 20× microscope objective (Nikon, Melville N.Y.). The slide containing the microarray is placed on a computer-controlled X-Y stage on the microscope and raster-scanned past the objective. The 1.8 cm×1.8 cm microarray used in the present example is scanned with a resolution of 20 micrometers.

Array elements that exhibited at least 2-fold change in expression at one or more time points, a signal intensity over 250 units, a signal-to-background ratio of at least 2.5, and an element spot size of at least 40% are identified as differentially expressed.

### Advantages and Business Applications

While the primary interest of this work is in developing better research tools for examining nutrient-gene interactions involved in type 2 diabetes mellitus (T2DM) and the renal complications which affect ˜40% of T2DM patients, almost every chronic disease is affected by one or more nutrients (see for example, Afman, L. and Muller, M. (2006) Nutrigenomics: from molecular nutrition to prevention of disease. J. Am. Diet. Assoc. 106: 569-576; Gross, J. L. et al. (2005) Diabetic nephropathy: diagnosis, prevention, and treatment. Diabetes Care 28: 164-176; and Kaput, J. et al. (2005) The case for strategic international alliances to harness nutritional genomics for public and personal health. Br. J. Nutr. 94: 623-632). Examples include salt and hypertension, saturated fats and cardiovascular disease, excess calories and obesity, and oxidative stress (hence antioxidants), inflammation, macronutrient calorie source and metabolic syndrome. This market model may be utilized by food ingredient manufacturers and consumer product companies for evaluation of specific ingredients, finished food products and combinations of foods intended for health maintenance and/or disease prevention or management. Likewise, dietary supplement providers, academic and industrial researchers, clinics, and hospitals associated with academic centers or privately held are envisioned as regular users of the facility for evaluation of dietary and lifestyle regimens intended for disease prevention or intervention and health maintenance.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1-56. (canceled)

57. A method for providing an individual with a personalized diet for said individual, the personalized diet optimised to reduce the likelihood that said individual will succumb to a disease or disorder, the method comprising the steps of (i) collecting nutritional information relating to one or more consumable items procured by the individual; (ii) storing the collected nutritional information in a first database; (iii) collecting biological information from the individual, which biological information is associated with health or disease outcomes; (iv) storing the collected biological information in a second database; (v) repeating steps (i) through (iv) at predetermined intervals; (vi) at any one interval, generating a statistical correlation that associates nutritional information with biological information; (vii) using the statistical correlation to provide the individual with a personalized diet optimised to reduce the likelihood of the individual succumbing to a disease or disorder; (viii) repeating any of steps (i) through (vii); the method providing the individual with a personalized diet for said individual.

58. The method of claim 57 wherein the biological information comprises data selected from the group consisting of: epigenetic data, genetic data, genomic data, nutrigenomic data, heart rate, breathing rate and volume, weight, BMI, HDL, LDL, cholesterol, glucose, lipids, HbA1c, blood pressure, and the presence or amount of biomarkers of inflammation, TNF-alpha, HsCRP, leukotrienes, prostaglandins, and hormones.

59. The method of claim 57 wherein the disease or disorder is selected from the group consisting of type 2 diabetes mellitus (T2DM), obesity, metabolic syndrome, Alzheimer's disease, cardiovascular disease, and cancer.

60. The method of claim 57 wherein the disease is diabetes and the biological information comprises measurements of glucose levels in the individual.

61. The method of claim 57 wherein, additionally, activity levels and calorie expenditure for individuals is measured and stored in a database, and correlated with nutritional information and biological information.

62. The method of claim 61 wherein activity levels are analyzed using dimensionality reduction algorithms and correlated with nutritional information and biological information.

63. A system for providing an individual with a personalized diet for said individual, the personalized diet optimised to reduce the likelihood that said individual will succumb to a disease or disorder, the system comprising: a computer loaded with a software operating system comprising an algorithm programmed to analyze a statistical correlation between two sets of data, the system further comprising performing the steps of (i) collecting nutritional information relating to one or more consumable items procured by the individual; (ii) storing the collected nutritional information as a component of a first database in the first read-write digital storage. device; (iii) collecting biological information from the individual; (iv) storing the collected biological information as a component of in a second database in the second read-write digital storage device; (v) repeating steps (i) through (iv) at predetermined intervals; (vi) at any one interval, using the computer to generate a statistical correlation between the nutritional information and the biological information; (vii) using the statistical correlation to provide the individual with a personalized diet optimised to reduce the likelihood of the individual succumbing to a disease or disorder; (viii) repeating any of steps (i) through (vii); the method providing the individual with a personalized diet for said individual.

64. The system of claim 63 wherein the biological information comprises epigenetic data, genetic data, genomic data, nutrigenomic data, measurements of heart rate, breathing rate and volume, blood pressure, weight, BMI, HDL, LDL, cholesterol, glucose, lipids, HbA1c, biomarkers of inflammation, TNF-alpha, HsCRP, leukotrienes, prostaglandins, and hormones.

65. The system of claim 63 wherein the disease or disorder is selected from the group consisting of type 2 diabetes mellitus (T2DM), obesity, metabolic syndrome, Alzheimer's disease, cardiovascular disease, and cancer.

66. The system of claim 63 wherein the disease is diabetes and the biological information comprises measurements of glucose levels in the individual.

67. The system of claim 63 wherein, additionally, activity levels and calorie expenditure for individuals is measured and stored in a database, and correlated with nutritional information and biological information.

68. The system of claim 67 wherein activity levels are analyzed using dimensionality reduction algorithms and correlated with nutritional information and biological information.

69. A research supermarket for determining personalized diets, the research supermarket comprising: a shop for selling food where unique product identifiers are associated with food items and are used to track and record data associated with such food items, and a computer loaded with a software operating system comprising an algorithm programmed to analyze a statistical correlation between two sets of data, the system further comprising performing the steps of (i) collecting nutritional information relating to one or more consumable items procured by the individual; (ii) storing the collected nutritional information as a component of a first database in the first read-write digital storage device; (iii) collecting biological information from the individual; (iv) storing the collected biological information as a component of in a second database in the second read-write digital storage device; (v) repeating steps (i) through (iv) at predetermined intervals; (vi) at any one interval, using the computer to generate a statistical correlation between the nutritional information and the biological information; (vii) using the statistical correlation to provide the individual with a personalized diet optimised to reduce the likelihood of the individual succumbing to a disease or disorder; (viii) repeating any of steps (i) through (vii); the method providing the individual with a personalized diet for said individual.

70. The research supermarket of claim 69 wherein the biological information comprises epigenetic data, genetic data, genomic data, nutrigenomic data, measurements of heart rate, breathing rate and volume, blood pressure, weight, BMI, HDL, LDL, cholesterol, glucose, lipids, HbA1c, biomarkers of inflammation, TNF-alpha, HsCRP, leukotrienes, prostaglandins, and hormones.

71. The research supermarket of claim 69 wherein the disease or disorder is selected from the group consisting of type 2 diabetes mellitus (T2DM), obesity, metabolic syndrome, Alzheimer's disease, cardiovascular disease, and cancer.

72. The research supermarket of claim 69 wherein the nutritional information collected is associated with one or more unique product identifiers physically associated with food items procured by the individual.
